(19) United States
(12) Patent Application Publication
(10) Pub. No.: US 2007/0280918 A1
(43) Pub. Date: Dec. 6, 2007

(54) Multitherapy Against Cancer

(76) Inventors: Laurent Schwartz, Paris (FR); Maurice Israel, Bures Sur Yvette (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/572,068

(22) PCT Filed: Jul. 12, 2005
(86) PCT No.: PCT/FR05/01797

§ 371 Date: Jul. 11, 2007

(30) Foreign Application Priority Data
Jul. 12, 2004 (FR) 0407752

(51) Int. Cl. A61K 38/54; A61P 35/00

(52) U.S. Cl. 424/94.2

(57) ABSTRACT

The invention concerns a pharmaceutical composition for treating cancer characterized in that it comprises a combination of a PP2A methylating agent and an active principle selected among the group consisting of a phosphatase PP1 inhibitor, a histone diacetylase inhibitor, a direct or indirect pyruvate kinase activator, a PTEN agonist, a tyrosine kinase inhibitor, a PI3 kinase inhibitor, a glucose competitor, a phosphocnol pyruvate carboxykinase inhibitor, a citrate synthase inhibitor and a latctate dehydro-genase inhibitor.

A subject of the present invention is a pharmaceutical composition intended for the treatment of cancer characterized in that it comprises the combination of at least several active ingredients chosen from the following: a PP2A methylating agent, a PP1 phosphatase inhibitor, a histone deacetylase inhibitor, a direct or indirect pyruvate kinase activator, a PTEN agonist, a tyrosine kinase inhibitor, a PI3 kinase inhibitor, a glucose competitor, a phosphoenol pyruvate carboxykinase inhibitor, a citrate synthase inhibitor and a lactate dehydrogenase inhibitor.

By “cancer” is usually meant a group of diseases having the appearance of tumours as symptoms. These tumours are composed of atypical cells, having a capacity for autonomous growth, an imprecise delimitation, an ability to invade neighbouring tissues and vessels and a tendency to disseminate by the production of metastases.

Although most of the factors promoting the appearance of tumours (genetic predispositions, exposure to radiation, substances, infections, age of the patients etc.) are known and a large number of genes involved in the development of cancer have now been identified, at present there is really no medicament which makes it possible to reverse the transformation of a tumour cell and to re-establish its normality.

The compositions used in chemotherapy are essentially intended to eliminate tumour cells from the organism, in particular when they are disseminated in the form of metastases. These compositions are more or less selective according to the type of cancer to be treated and often poorly tolerated by the organism. These compositions cannot therefore be prescribed over a long period without having consequences aggravating the patients' state of health. Moreover, they have the drawback of not completely preventing the recurrence of the cancer since the origin of the cancer at cell level is not treated.

In reality the therapeutic treatment of cancer comes up against the absence of clear, well-characterized therapeutic targets, common to the different types of cancer. If such targets had been identified without doubt, effective compounds having a real power of remission for patients would already have been identified.

Numerous compounds are mentioned in the literature as having cytostatic effects. However these compounds, taken in isolation, have not thus far made it possible to provide definitive care for cancer patients.

As a result of this situation, surgery, sometimes complemented by chemotherapy, remains the most effective way of treating cancer.

Faced with this fact, the inventors have had the idea of designing a multitherapy based on a phenomenological approach to cancer, which could complemented or replace the existing cancer therapies (surgery, radiotherapy, chemotherapy etc.).

The inventors based their work on the observations made by Otto Warburg (1883-1970), before the Second World War, according to which tumour cells produce more lactic acid than healthy cells, and consume increased quantities of glucose [Watson J. D., Molecular biology of the gene, 3d Ed., W. A. Benjamin Inc Menolo Park Calif., 1976].

They interpreted the overproduction of lactic acid and the increased consumption of glucose as being the consequence of a deregulation of glycolysis (Embden-Meyerhof pathway).

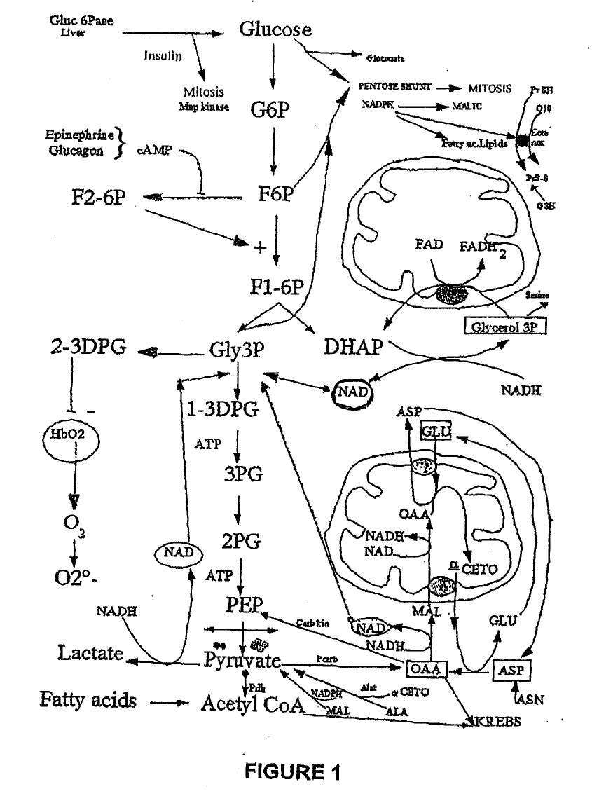

Glycolysis is defined as a series of reactions which involves degrading glucose-6-phosphate in order to form pyruvate and makes it possible to provide the cell with energy (two ATP molecules) as well as a certain reducing potential (NAD reduction). This series of reactions uses the action of various enzymes [LEHNINGER A. et al. Principes de biochimie, FLAMMARION. 1994], in particular pyruvate kinase, which is the only enzyme allowing the conversion of phosphoenol pyruvate (PEP) to pyruvate (FIG. 1).

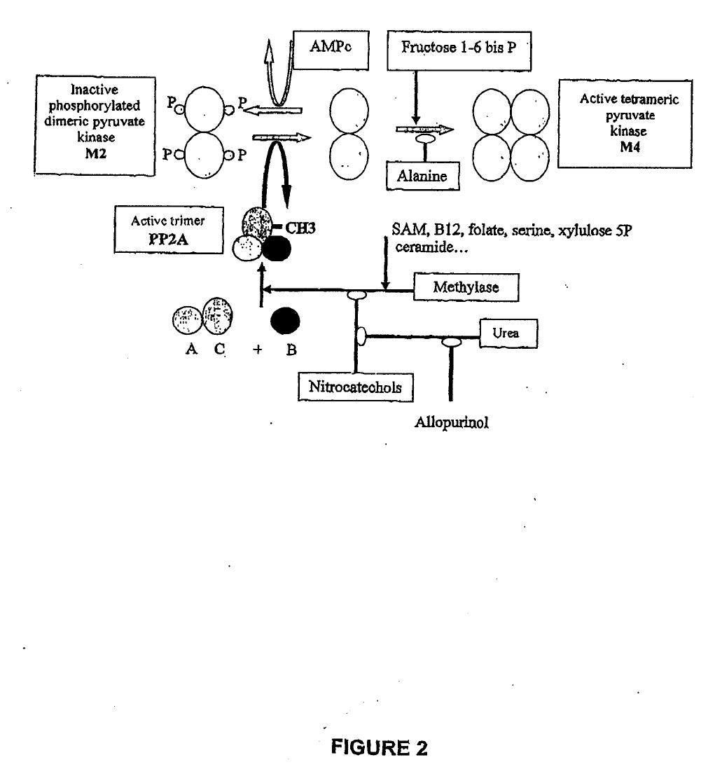

It has been demonstrated in the case of certain cancers, that pyruvate kinase could be kept inactive in a phosphorylated dimeric form (M2), whilst the active form of this enzyme is dephosphorylated tetrameric (M4) (FIG. 2).

When pyruvate kinase is present in its inactivated form M2, the formation of pyruvate by the Embden-Meyerhof pathway is interrupted. The cell in these circumstances must face both the accumulation in the cytoplasm of glucose-6-P degradation products, such as fructose 1-6P, fructose 6P, phosphoenol pyruvate, Gly-2,3 biphosphate (2-3 DPG) as well as other glucose metabolites, and a pyruvate deficit.

The accumulation of 2-3 DPG in the cytoplasm is harmful to the cell as the 2-3 DPG increases the release of oxygen by the haemoglobin, which forms tissue superoxide. Such a production of free radicals has a destructive effect on numerous cell constituents, in particular the DNA, which causes mutations in the genes.

The pyruvate deficit, for its part, obliges the cell to mobilize malic acid and alanine so as to form pyruvate by a metabolic pathway other than Embden-Meyerhof, which results in a significant production of lactate converting to lactic acid.

The accumulation of lactic acid has negative effects on the cell as it tends to acidify the cell medium, which, by the destruction of certain tissues, promotes the diffusion of the cancer [Stern R. et al., Lactate stimulates fibroblast expression of hyaluronan and CD44: the Warburg effect revisited].

In parallel, the beta-oxidation of fatty acids results in the production of acetyl coA. However this production consumes energy and is insufficient to meet the needs of the cell.

The reduction in the quantity of pyruvate available in the cell leads to a deficit of acetyl-CoA, which is the main substrate of the Krebs cycle. The main means of compensating for this acetyl-CoA deficit for the cell is to promote the entry of glucose into the cytoplasm. The increase in the glucose level that is observed in cancer cells is explained by this paradoxical compensation phenomenon.

It is, moreover, well known that the entry of glucose into the cell is positively modulated by the insulin receptors which are coupled with the activation of certain effectors, in particular the PI3 kinases, which have the capacity to inhibit PTEN phosphatase (see below) and the MAP kinases.

The MAP-kinases have the characteristic of being able to activate phosphatase PP1, which is one of the main activators of the MPF (Maturation Promoting Factor).

The MPF is a complex combining kinases and cyclins which allows the cell to pass from one phase of the cell cycle to another and thus leads to mitosis. Mitosis is defined as the division of a mother cell into two daughter cells.

When the cells are in mitosis, they are faced with significant energy needs, which obliges them to accentuate their glucidic-type metabolism. In the case of a glycolysis dysfunction, such as that just mentioned, the production of lactate and free radicals is systematically increased, whilst the cell does not provide the quantities of acetyl-CoA necessary for the Krebs cycle. The cell is therefore forced to allow still more glucose to enter, which has the double consequence of stimulating cell division and increasing the metabolic disorder within the cell.

Under such conditions, after several cell cycles, the cell divisions become increasingly anarchic. The cells lose their character of differentiated cells in order to be transformed into poorly differentiated tumour cells having the ability to migrate to other parts of the body and capable of becoming the focus of new tumours.

Having thus observed that the canceration process could be explained by a deficiency in glycolysis, in particular a deficiency in the enzyme pyruvate kinase, the inventors sought means of re-establishing, by different means, the normal functions of the Embden-Meyerhof pathway in cancer cells.

They developed pharmaceutical compositions comprising active ingredients intended to reduce the accumulation of glycolytic products in the cell, in particular for the purpose of re-establishing the enzymatic activity of the pyruvate kinase.

The combination of several of these active ingredients makes it possible to obtain pharmaceutical compositions having the double function of regulating glycolysis, but also of limiting the entry of glucose into the cell.

A more particular subject of the invention is compositions comprising the combination of a phosphatase PP2A methylating agent with one or more of the abovementioned active ingredients such as a PTEN agonist, a lactate dehydrogenase inhibitor, or a phosphoenol pyruvate carboxykinase inhibitor.

Unlike the therapies of the prior art, the present compositions are intended, above all, to inhibit the conversion process of the tumour cells.

### DESCRIPTION

Generally, the present invention relates to a pharmaceutical composition intended for the treatment of cancer characterized in that it comprises one or more active ingredients having the direct or indirect effect of regulating glycolysis or reducing the entry of glucose into the cell.

The active ingredients covered by the invention are the following: a PP2A methylating agent, a PP1 phosphatase inhibitor, a histone deacetylase inhibitor, a direct or indirect pyruvate kinase activator, a PTEN agonist, a tyrosine kinase inhibitor, a PI3 kinase inhibitor, a glucose competitor, a phosphoenol pyruvate carboxykinase inhibitor, a citrate synthase inhibitor and a lactate dehydrogenase inhibitor.

Said active ingredients can be administered simultaneously or successively over time.

A pharmaceutical composition according to the invention provides more particularly the combination of at least two of these active ingredients.

According to a preferred aspect of the invention, the combination of these active ingredients comprises, on the one hand, at least one PP2A methylating agent, and on the other hand, at least one active ingredient chosen from the group comprising a PP1 phosphatase inhibitor, a histone deacetylase inhibitor, a direct or indirect pyruvate kinase activator, a PTEN agonist, a tyrosine kinase inhibitor, a PI3 kinase inhibitor, a glucose competitor, a phosphoenol pyruvate carboxykinase inhibitor, a citrate synthase inhibitor and a lactate dehydrogenase inhibitor.

Such a composition is intended to act on the essential stage of glycolysis (Embden-Meyerhof pathway) which involves the conversion of PEP (Phosphoenol pyruvate) to pyruvate with the formation of ATP. This stage which has no biochemical replacement pathway, is catalyzed by an enzyme, pyruvate kinase, which is naturally present in two forms: a tetrameric form (M4) which corresponds to its active form and a dimeric form (M2) corresponding to the inactive form (FIG. 2). Studies have shown that the active form (M4) corresponded to the dephosphorylated form of the enzyme.

In certain cancer cases, the conversion of PEP (Phosphoenol pyruvate) to pyruvate by pyruvate kinase does not take place properly. This defective conversion results in an accumulation of glycolytic products in the cytoplasm of the cell, having as a consequence the effects on the metabolism described by Warburg. Studies [Mazurek S. et al., 2002, pyruvate linase type M2: a crossroad in the tumor metabolome, Br. J.Nutr., 87suppl1:S23-9] have shown that the low yield of conversion of the PEP to pyruvate, in the case of these cancers, was correlated with an insufficient quantity of enzyme of the dephosphorylated active form M4 compared with those of the phosphorylated form M2.

It is therefore apparent that a phosphatase capable of converting the pyruvate kinase from the form M2 to the form M4 was required for this essential stage of glycolysis, which involves converting the PEP to pyruvate. By phosphatase is meant an enzyme capable of releasing phosphoric acid in order to activate the pyruvate kinase.

The inventors have identified the phosphatase in question as phosphatase PP2A or a protein with an equivalent function.

Phosphatase PP2A is described by Vafai and Stock [2002, Protein phosphatase 2A methylation: a link between elevated plasma homocysteine and Alzheimer's disease, FEBS Lett, 8: 518: 1-4] as being involved in the dephosphorylation of the Tau proteins, which are proteins involved in the formation of the mitotic spindle. The Tau proteins have the capacity to precipitate the tubulin of the microtubules which make up the mitotic spindle. The works of Vafai and Stock have shown that the activation of PP2A required the intervention of carboxy-methylase which has the function of methylating PP2A.

In Alzheimer's disease, a methyl deficit thus renders PP2A incapable of repressing the Tau proteins. In the absence of methylated PP2A, the Tau proteins are hyper-phosphorylated which has the consequence of abnormal polymerization of the tubulin inside the neurons. The polymerized tubulin then forms plaques inside the neurons, which causes neuron dysfunction.

Phosphatase PP2A is described as having different functions, one of which involves maintaining the MPF (Maturation Promoting Factor) in an inactive form. The MPF is a cell cycle regulator combining kinases and cyclins in the form of a complex. When PP2A is found in its active, i.e. methylated, form it represses the activation of the MPF by dephosphorylating one of the proteins of the complex called CDC25. As in the case of the Tau proteins, PP2A must be suitably methylated in order to be able to effectively repress CDC25 [Karaiskou A. et al. 1999, Phosphatase 2A and polo kinase, two antagonist regulators of cdc25 activation and MPF auto amplification, J. cell. ScL, 112:3747-56]. Thus, when PP2A is not suitably activated or defective, CDC25 is phosphorylated by the action of another enzyme: CDC25 phosphorylase. In a similar case, the phosphorylation of CDC25 leads to the activation of the MPF and to the triggering of a new phase of the cell cycle leading to mitosis.

Phosphatase PP2A therefore exerts a power of regulation both on the regulation of glycolysis, on the formation of the mitotic spindle (via the Tau proteins), and on the cell cycle (via CDC25).

Phosphatase PP2A is also known to have an activity in the phosphorylation of tumour-suppressing genes such as rb or p53 [Li X. et al., 2001, Protein Phosphatase 2A and its B56 regulatory subunit inhibit Wnt signaling in Xenopus, EMBO J. 20:4122-31]. This activity constitutes an additional reason for maintaining PP2A at a high level of expression (or enzymatic activity) in cancer patients.

A preferred feature of the invention therefore involves proposing a pharmaceutical composition for the treatment of cancer comprising at least one active ingredient having the function of maintaining or restoring the activity of PP2A.

Phosphatase PP2A has, in its natural state, numerous isomorphs [Lechward K. et al., 2001, Protein phosphatase 2A: variety of forms and diversity of functions, Acta. Biochim. Pol., 48:921-33] and a genetic polymorphism capable of explaining certain variations observed in the development of cancer. Such polymorphism is very useful in the diagnosis of cancer, the identification of new therapeutic targets and the determination of the active sites of the enzyme PP2A.

This is why a particular feature of the invention is the use of the nucleotide sequences of the genes coding for PP2A or one of its isoforms, for the purposes of genotyping or diagnosing cancer.

The peptide sequences corresponding to the abovementioned nucleotide sequences, obtained by recombinant route or extracted from samples taken from a patient, can be used for diagnostic purposes, in particular with a view to producing immunological tests.

Antibodies specifically recognizing the peptide sequences of PP2A can, moreover, be obtained, not only for the purposes of producing these immunological tests, but also for a therapeutic purpose.

Another particular feature of the invention involves analyzing the way in which PP2A is methylated or de-methylated on contact with different substances, in order to determine the carcinogenic activity of a real or potential toxic substance.

A more preferred feature of the invention is a pharmaceutical composition for the treatment of cancer, comprising at least one PP2A methylating agent.

By methylating agent is meant any compound making it possible to transfer a methyl group to PP2A.

The preferred methylating agents of the invention are serine, folate, methionine, S-adenosyl methionine, vitamin B12, tetrahydrofolate, choline, acetylcholine manganochloride and betaine.

A particularly preferred PP2A methylating agent of the invention is betaine (betaine hydrate or also trimethylammonio-2 acetate) or one of its pharmaceutically acceptable salts, in particular betaine citrate.

More generally, the methylating agents are described in the literature as compounds having a positive influence with respect to the prevention of cancer. Epidemiological studies have thus shown that the consumption of methyl-rich products tended to reduce the risks of cancer [Pascale R. M. et al., 2002, Chemoprevention of hepatocarcinogenesis: S-adenosyl-L-methionine, Alcohol 27:193-8].

However certain authors consider that an excess of methyl donor can increase the risks of lung cancer, lymphomas and leukaemia. They have shown, in fact, that the excess of methyl at nucleus level could lead to a hypermethylation of the promoting sequences of genes coding for essential proteins, in particular the PTEN phosphatases (see below) which are considered as proteins having anti-tumour effects [Soria J. C. et al., 2002, Lack of PTEN expression in non small cell lung cancer could be related to promoter methylation, Clin. Cancer. Res., 8:1178-84]. Moreover, certain treatments for cancer use as active ingredients methotrexate and aminopterine, which both have the effect of inhibiting the methylation reactions.

The invention also proposes pharmaceutical compositions combining a methylating agent with at least one other active ingredient among those mentioned previously making it possible to limit the side effects of an excess of methyl in the cell, such as in particular a histone deacetylase inhibitor.

Histone-deacetylases are enzymes which fix the methylated groups to the gene promoting sequences in order to prevent their transcription. The use of inhibitors of these enzymes has the purpose of limiting the excessive “silencing” of the genes, brought about by too high a contribution of methylated groups at nucleus level.

By “inhibitor” is meant in the present document, a molecular effector capable of reducing the activity of a given enzyme.

The preferred inhibitors of histone deacetylase of the invention are butyrate, phenylbutyrate, trichostatin and valproate, or a combination thereof.

It is also envisaged to add a PTEN protein agonist to the pharmaceutical compositions according to the invention.

By PTEN protein agonist is meant a natural or medicamentous substance having, at least in part, the same effects as the PTEN proteins.

Studies have shown that the PTEN proteins were repressed in certain cancer cell lines.

These proteins are described as negatively regulating the type PI3 kinases involved in the transport of glucose. It is therefore useful to maintain a high level of activity of the PTEN proteins in order to limit the entry of glucose into the cell or to use an agonist making it possible to obtain the same effects.

A preferred PTEN protein agonist of the invention is rosiglitazone [Patel L. et al., 2001, tumor suppressor and anti-inflammatory actions of PPARgamma agonists are mediated via upregulation of PTEN, Current. Biol. 11:764-8].

A preferred feature of the invention is a composition comprising a combination of a PTEN protein agonist such as rosiglitazone, and a PP2A methylating agent such as betaine citrate.

The effect of reducing the entry of glucose into the cells can be obtained by products other than those mentioned above. In particular products known to be useful in the treatment of diabetes can be used. These products, to the extent that their combination with the other active ingredients of the invention is not toxic to patients, are considered as being able to be included in a pharmaceutical composition according to the invention.

A preferred feature of the invention therefore involves combining at least one methylating agent with at least one product useful in the treatment of diabetes for the purpose of treating cancer.

One possibility, for example, is to add to the pharmaceutical compositions according to the invention one or more analogues or competitors of glucose, such as 5-mannoheptulose or 2-deoxy-glucose.

By analogue is meant a molecule with a similar structure which can be substituted by another molecule.

By competitor is meant an analogue capable of competing with said molecule on its action site.

It is also possible to combine active ingredients described previously with tyrosine kinase inhibitors.

Kinases are enzymes which ensure the transfer of a phosphate originating from adenosine triphosphate (ATP) to an acceptor which is thus activated.

The tyrosine kinase inhibitors make it possible to inhibit the action of the insulin receptors to tyrosine kinase. They make it possible in particular to repress the activity of the tyrosine kinases, so as to inhibit the MAP kinase signalling pathway, on which the activity of the PP1 phosphatase depends.

The preferred tyrosine kinase inhibitors of the invention are PD 9805G, adenosine or actual anti-cancer medicaments such as GLIVEC or IRESSA.

PI3-kinase (phosphatidyl-inositol 3-kinase) is an enzyme by means of which the insulin receptors control the entry of glucose into the cell. The use of PI3-kinase inhibitors in a composition according to the invention is intended to limit the entry of sugars, in particular that of glucose, into the cell cytoplasm.

The PI3-kinase inhibitors preferred within the framework of the invention are wortmannin, LY294002, quercetin, myricetin and staurosporine.

PP1 phosphatase is an enzyme which, unlike PP2A, has the capacity to activate CDC25. The activation of CDC25 has as a consequence the activation of the MPF, which as described previously is one of the main factors in triggering mitosis. PP1 therefore acts in the opposite direction to PP2A. When PP2A is deficient, the cell cycle is activated and the cell is found in a situation where the cell cycle is no longer controlled.

According to a preferred aspect of the invention, the pharmaceutical compositions of the invention include the combination of at least one of the active ingredients described previously with a PP1 inhibitor for the purpose of reducing the effects of PP1 on the cell cycle. These inhibitors can be specific or non-specific inhibitors [Yan Y et al., 1999, distinct roles for PP1 and PP2A in phosphorylation of the retinoblastoma protein. PP2A regulates the activity of G(1) cyclin dependant kinases. J. Biol. Chem., 274(3):1917-24].

The preferred PP1 inhibitors according to the invention are chosen from cantharidin, tautomycin and rapamycin [Mc Clusey A. et al., 2001, The inhibition of protein phosphatases 1 and 2A: a new target for rational anticancer drug design?, Anticancer Drug. Des., 16:291-303] which are fairly wide-spectrum phosphatase inhibitors, or the combination of one of these inhibitors. PP1 can also be inhibited via its natural antagonist DARP 32, which is activated by the D1 agonists such as dopamine. It is, of course, suitable to dose this inhibitor so as to inhibit PP1 while retaining the functionality of PP2A.

According to a particular feature of the invention, a PP1 inhibitor can be used only for the purposes of producing a medicament for limiting the progression of the cell cycle.

According to another feature of the invention, a direct or indirect pyruvate kinase activator such as fructose 1-6 biphosphate or xylulose-5P [Nishimura M. et al., 1995, Purification and characterization of a novel xylulose 5-phosphate activated protein phosphatase catalyzing dephosphorylation of fructose-6-phosphate, 2-kinase:fructose-2,6-biphosphatase, J. Biol. Chem., 270:26341-6] is used to activate the conversion reaction of the PEP to pyruvate during glycolysis.

A pyruvate kinase activator according to the invention makes it possible to increase the yield of the reaction catalyzed by the pyruvate kinase, in particular when PP2A is deficient in its pyruvate kinase activation role. An activator is direct when it acts as activator directly on the pyruvate kinase, it is indirect when it acts on the pyruvate kinase via an intermediate activator, for example PP2A.

Preferably, the pyruvate kinase activator used for the production of a medicament intended for the treatment of cancer is, depending on choice, a xylulose 5-P, a ceramide, an agonist of the A1 adenosine receptors such as N-6-cyclopentyladenosine, a manganese salt such as acetylcholine manganochloride or a combination of these products. The purpose of these activators is to avoid the phenomenon of glycolysis saturation which is observed in the Warburg effect.

A composition according to the invention can moreover comprise one or more phosphoenol pyruvate carboxykinase inhibitors and one or more citrate synthase inhibitors.

In the case of cancer, it is assumed that these two enzymes, citrate synthase and phosphoenol pyruvate carboxykinase remain active.

Phosphoenol pyruvate carboxykinase is an enzyme which is involved in cancer in the conversion of phosphoenol to oxalo-acetate. By means of the action of citrate synthase, this oxaloacetate is converted with acetyl-coA to citrate in the first stage of the Krebs cycle. The inhibition of phosphoenol pyruvate carboxykinase and citrate synthase thus reduce the catabolism in particular of the fatty acids and amino acids.

Preferred phosphoenol pyruvate carboxykinase inhibitors are aspartate, mefformin, chlorophosphoenol pyruvate and the derivatives of tryptophan. A citrate synthase inhibitor can be fluoroacetyl co-A.

A preferred feature of the invention is a composition comprising the combination of a phosphoenol pyruvate carboxykinase inhibitor, such as metformin or chlorophosphoenol pyruvate, and a PP2A methylating agent such as betaine citrate.

Another feature of the invention is the use of chlorophosphoenol pyruvate as a therapeutic agent for the treatment of cancer. This use makes it possible, in particular, to inhibit the activity of phosphoenol pyruvate carboxykinase.

The combination of chlorophosphoenol pyruvate and carnitine, a substance which makes it possible to target the mitochondria, is advantageous for the treatment of cancer.

A molecule which is a chlorophosphoenol pyruvate and carnitine ester, in which carnitine and chlorophosphoenol pyruvate are covalently linked, has proved to be particularly effective for inhibiting the growth of cancer cell lines.

The present invention therefore covers a pharmaceutical composition making it possible to treat cancer comprising chlorophosphoenol pyruvate and, optionally, carnitine, as well as any molecule being a chlorophosphoenol pyruvate and carnitine ester which can be included in such a composition.

A composition according to the invention can also comprise a lactate dehydrogenase inhibitor as an active ingredient.

Lactate dehydrogenase is an enzyme which is involved in cell metabolism in the degradation of pyruvate to lactate. It therefore participates in a reduction in the store of pyruvate in the cell and an increase in lactate. Alanine, which represents 20 to 30% of the amino acids present at muscle level can be mobilized to form pyruvate and therefore, if appropriate, lactate.

A preferred lactate dehydrogenase inhibitor is a derivative of alanine, more particularly bromo-alanine, or one of its derivatives or analogues. The inhibition can be carried out, for example, by placing one of these compounds in competition with alanine at the level of the catalytic site of the alanine dehydrogenase, the enzyme which allows the conversion of alanine to pyruvate. There is then a reduction in the conversion of alanine to pyruvate and therefore a reduction in the quantity of substrate available for the lactate dehydrogenase.

A bromo-alanine analogue is defined as being a compound the structure of which is close to that of bromo-alanine, to the point that it makes it possible to inhibit alanine dehydrogenase in the same manner as bromo-alanine.

The pharmaceutical compositions according to the invention are useful for the preparation of a medicament intended for the treatment of cancer. They can be used in a method for the treatment of a cell growth abnormal in a mammal, in particular when the abnormal cell growth is a cancer, more particularly forming part of the group comprising lung cancer, bone cancer, pancreatic cancer, skin cancer, head and neck cancer, a cutaneous or intraocular melanoma, uterine cancer, ovarian cancer, cancer of the rectum, cancer of the anal region, stomach cancer, cancer of the colon, breast cancer, a carcinoma of the Fallopian tubes, a endometrial carcinoma, a carcinoma of the cervix, a carcinoma of the vagina, a carcinoma of the vulva, Hodgkin's disease, cancer of the oesophagus, cancer of the small intestine, endocrine system cancer, thyroid cancer, parathyroid gland cancer, adrenal gland cancer, a soft tissue sarcoma, urethral cancer, penile cancer, prostate cancer, chronic or acute leukaemia, lymphocytic lymphomas, bladder cancer, cancer of the kidney or ureter, a renal cell carcinoma, a renal pelvis carcinoma, tumours of the central nervous system (CNS), a primary lymphoma of the CNS, spinal cord tumours, a brain stem glioma, a hypophyseal adenoma or a combination of several of the abovementioned cancers.

The pharmaceutical compositions according to the invention can take different forms, for example, solids or liquids and be presented in the pharmaceutical forms commonly used in human medicine, such as for example plain or sugar-coated tablets, gelatin capsules, granules, caramels, suppositories, injectable preparations; they are prepared according to the usual methods. The active ingredient or ingredients can be incorporated into excipients usually used in these pharmaceutical compositions, such as talc, gum arabic, lactose, starch, magnesium stearate, cocoa butter, aqueous or non-aqueous vehicles, fats of animal or vegetable origin, paraffinic derivatives, glycols, various wetting agents, dispersants or emulsifying agents, preservatives.

A subject of the present invention is also a method for the preparation of a composition described above, characterized in that, according to methods known per se, the active ingredient or ingredients are mixed with acceptable, in particular pharmaceutically acceptable, excipients.

The invention also consists of a product containing at least one PP2A methylating agent, and on the other hand, at least one active ingredient chosen from the group comprising a PP1 phosphatase inhibitor, a histone deacetylase inhibitor, a direct or indirect pyruvate kinase activator, a PTEN agonist, a tyrosine kinase inhibitor, a PTEN agonist, a tyrosine kinase inhibitor, a PI3-kinase inhibitor, a glucose competitor, a phosphoenol pyruvate carboxykinase inhibitor and a citrate synthase inhibitor, as combination products for a simultaneous or separate use or use spread over time in cytostatic or anti-cancer therapy.

A particularly preferred feature of the invention is the use of xylulose 5-P for the production of a medicament intended for the treatment of cancer.

FIG. 1: Simplified diagram of the glucose metabolism in the cell, showing the main stages of glycolysis. The dominant position occupied by pyruvate kinase in the process leading to the production of pyruvate and acetyl CoA, which are essential to the Krebs cycle, will be noted.

FIG. 2: Activation of pyruvate kinase by PP2A. This figure shows how phosphatase PP2A, in the form of a trimer, is activated by the action of a methylase using as methyl donors, compounds such as vitamin B12, folate and serine and, as secondary activators, other compounds such as xylulose-5P and ceramide. This methylation renders PP2A active vis-à-vis pyruvate kinase. Once methylated, PP2A allows the activation of the pyruvate kinase by causing it to pass from its inactive phosphorylated form M2 to its active dephosphorylated form M4. This reaction consumes Fructose 1-6 bis P.

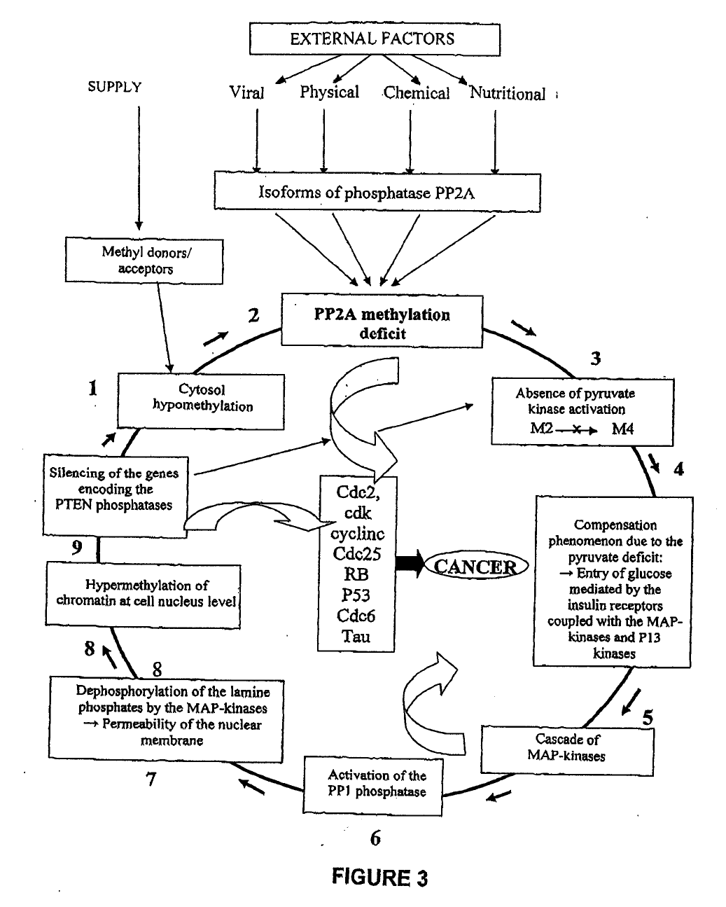

FIG. 3: Incidence of different factors (virus, physical or chemical and nutritional action) on the integrity of PP2A and involvement of a PP2A deficiency in the canceration process. This figure reconstitutes the process by which a PP2A deficiency is at the origin of a canceration process taking the form of a “vicious circle”. The inventors have reconstituted the sequence of the different stages of this process from sparse bibliographic data. It is from this overall diagram that the pharmaceutical compositions according to the invention have been defined. The starting point of this diagram is alteration by exogenous factors of the enzymatic activity of phosphatase PP2A. This alteration has the consequence of inactivation of pyruvate kinase and therefore a reduction in the glycolysis yield. The cell compensates for this reduction in glucose degradation yield by an additional supply of glucose. This entry of glucose leads, on the one hand, to the activation of the MAP-kinases and PI13-kinase, which are linked to the insulin receptors tyrosine kinase, and on the other hand, to a glycolysis saturation. The MAP-kinases activate the PP1 phosphatase which acts positively on certain cell cycle regulators, promoting mitosis. In parallel, the MAP-kinases increase the permeability of the nuclear membrane, which allows methyl to enter into the nucleus, having the effect of hypermethylation of the genes, in particular those encoding the PTEN phosphatases. When the activity of the PTEN phosphatases is reduced, the PI3-kinase signalling pathway is increased and the entry of glucose into the cell becomes all the greater. Finally, the cell experiences a metabolic disorder such that its development becomes completely anarchic.

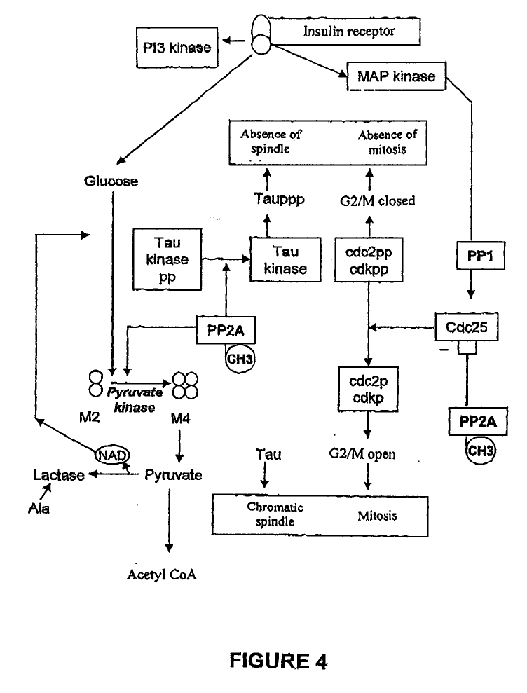

FIG. 4: Diagram showing the different fields of intervention of PP2A and PP1 and their respective regulation modes. This diagram, produced by the inventors, makes it possible to visualize the strength of the relationship existing between the PP2A and PP1 phosphatases. Their action is opposite overall, which explains that it is necessary to support the activity of PP2A while repressing that of PP1. PP2A both acts on glycolysis via the activation of pyruvate kinase and inhibits the cell cycle by repressing CDC25. It is therefore a factor tending to maintain the cell in a differentiation phase. Conversely, PP1 acts positively on CDC25 and acts as a mitogenic agent. PP1 is activated by the MAP-kinases and therefore reacts positively to the entry of glucose into the cell.

The embodiment examples which follow are intended to illustrate the invention. They have no limitative character.

### Example 1

### Betaine Citrate/Rosiglitazone Combination

Compositions comprising different concentrations of a PP2A methylating agent (betaine citrate) and a known PTEN agonist (rosiglitazone) were tested in-vitro in parallel on the following 3 lung cancer cell lines: Calu-6 and NCI-H460.

The rosiglitazone used is that marketed by GlaxoSmithKline in the preparation sold under the name of Avandia®.

1) Prior determination of the IC50 of the products taken in isolation on the cell lines.

The result of the preliminary tests aimed at determining separately the effect of betaine citrate and rosiglitazone on the different cell lines, are given in Table 1 below.

TABLE 1
Average and standard deviation (SD) of IC₅₀ recorded for betaine
citrate on the cell lines Calu-6 and NCI-H460.

IC₅₀ (μM)
Betain citrate

| Cell lines | Average | SD |
|---|---|---|
| Calu-6 | 2.17 | 0.56 |
| NCI-H460 | 1.49 | 0.49 |

TABLE 1
Average and standard deviation (SD) of IC₅₀ recorded for
Avandia ® on the cell lines Calu-6 and NCI-H460.

IC₅₀ (μM)
Avandia ®

| Cell lines | Average | SD |
|---|---|---|
| Calu-6 | 14.05 | 3.42 |
| NCI-H460 | 8.84 | 0.68 |

It should be noted that the cell lines tested are less sensitive to betaine citrate (IC₅₀ of the order of a millimol), but not vis-à-vis rosiglitazone (IC₅₀ of the order of ten micromols).

2) Combination of betaine citrate (NaBt) and rosiglitazone:

Different concentration ratios were established, which were tested on the different cell lines.

The results of these experiments are presented in Tables II, III and IV below. The synergy index (CI) which is represented by the letters CI (combination index) in the right-hand column of the different tables was calculated according to the method described by Chou and Talalay [Chou, T.C. et al. in Encyclopaedia of Human Biology, Academy Press (1991) 2:371-379].

When CI>1, it must be considered that the products which are combined have an antagonistic effect;

When CI=1, it must be considered that the products which are combined have a simple additive effect;

When CI<1, it must be considered that the products which are combined have a synergistic effect;

For each cell line three tests were carried out.

TABLE 3
Noted effect of NaBt and Avandia ® on the growth of Calu-6 cells.

| Treatment | | | | | | |
|---|---|---|---|---|---|---|
| [Avandia] | [NaBt] | Exp 1 | Exp 2 | Exp 3 | | |
| (μM) | (mM) | CI | CI | CI | Average | SD |
| 0.1 | 0.10 | 0.06 | 0.38 | 0.01 | 0.15 | 0.20 |
| 0.5 | 0.25 | 0.14 | 0.05 | 0.01 | 0.07 | 0.07 |
| 1.0 | 0.50 | 0.07 | 0.05 | 0.06 | 0.06 | 0.01 |
| 5.0 | 1.00 | 0.04 | 0.08 | 0.13 | 0.08 | 0.05 |
| 10.0 | 2.00 | 0.11 | 0.17 | 0.38 | 0.22 | 0.14 |

TABLE 4
Noted effect of NaBt and Avandia ® on
the growth of NCI-H460 cells.

| Treatment | | | | | | |
|---|---|---|---|---|---|---|
| [Avandia] | [NaBt] | Exp 1 | Exp 2 | Exp 3 | CI | |
| (μM) | (mM) | CI | CI | CI | Average | SD |
| 0.1 | 0.10 | 0.50 | 0.20 | 0.23 | 0.31 | 0.17 |
| 0.5 | 0.25 | 0.86 | 0.16 | 0.36 | 0.46 | 0.36 |
| 1.0 | 0.50 | 0.75 | 0.17 | 0.17 | 0.36 | 0.33 |
| 5.0 | 1.00 | 0.56 | 0.16 | 0.06 | 0.26 | 0.26 |
| 10.0 | 2.00 | 0.38 | 0.07 | 0.24 | 0.23 | 0.16 |

These results show that a better inhibition of the growth of the cancer cell lines Calu-6 and NCI-H460 is obtained when the two products are used in combination, than when they are used separately.

In parallel, a study of the toxicity of the products on mice was carried out. No mortality or weight loss was observed for the treatment combining betaine citrate and Avandia®.

### Example 2

### Betain Citrate/Metformin Combination

The same protocol was used as in Example 1 to test the synergy between a PP2A methylating agent (betaine citrate) and a known phosphoenol pyruvate carboxykinase inhibitor which is metformin.

The cell line adopted for carrying out the experiment is the line Calu-6.

The protocol used is that described by Chou and Talalay mentioned above, which here requires a determination of the fraction affected (Fa), for each of the products betaine citrate and mefformin, taken in isolation then in combination. These Fa values are used in the calculation of the synergy index (CI) which makes it possible to report the presence or absence of synergy between the products.

TABLE 5
Individual determination of the fraction affected (Fa) for the
products betaine citrate (NaBt) and metformin (Met)
on the lung cell line Calu-6.

Treatment

| [NaBt] | [Met] | Fa | | |
|---|---|---|---|---|
| (mM) | (mM) | Exp 1 | Exp 2 | Exp 3 |
| 0.05 | — | 0.00 | — | — |
| 0.10 | — | 0.00 | 0.02 | 0.00 |
| 0.13 | — | 0.03 | — | — |
| 0.20 | — | 0.12 | — | 0.00 |
| 0.25 | — | 0.02 | 0.08 | 0.00 |
| 0.50 | — | 0.31 | 0.15 | 0.26 |
| 1.00 | — | 0.59 | 0.18 | 0.63 |
| 2.00 | — | 0.69 | 0.37 | 0.77 |
| 4.00 | — | 0.77 | 0.61 | 0.83 |
| — | 0.05 | 0.03 | — | — |
| — | 0.10 | 0.08 | — | 0.04 |
| — | 0.13 | 0.08 | — | — |
| — | 0.20 | 0.04 | 0.04 | 0.06 |
| — | 0.25 | 0.06 | 0.06 | 0.08 |
| — | 0.50 | 0.08 | 0.09 | 0.13 |
| — | 1.00 | 0.18 | 0.19 | 0.25 |
| — | 2.00 | 0.03 | 0.31 | 0.36 |
| — | 4.00 | 0.36 | 0.40 | 0.46 |

TABLE 6
Determination of the fraction affected (Fa) and of the corresponding
synergy index (CI) for the products betaine citrate (NaBt) and
metformin (Met) in combination on lung cell line Calu-6. Different
concentration ratios were used (1:1, 1:2, 2:1).

| [NaBt] | [Met] | Exp 1 | | Exp 2 | | Exp 3 | |
|---|---|---|---|---|---|---|---|
| (mM) | (mM) | Fa | CI | Fa | CI | Fa | CI |

| ratio 1:1 | | | | | | | |
|---|---|---|---|---|---|---|---|
| 0.10 | 0.10 | 0.02 | 4.49 | 0.03 | 1.58 | 0.02 | 2.38 |
| 0.25 | 0.25 | 0.08 | 1.58 | 0.07 | 1.64 | 0.16 | 0.69 |
| 0.50 | 0.50 | 0.35 | 0.64 | 0.31 | 0.57 | 0.46 | 0.50 |
| 1.00 | 1.00 | 0.61 | 0.72 | 0.51 | 0.50 | 0.73 | 0.56 |
| 2.00 | 2.00 | 0.67 | 1.27 | 0.64 | 0.60 | 0.79 | 0.97 |

| ratio 1:2 | | | | | | | |
|---|---|---|---|---|---|---|---|
| 0.10 | 0.20 | 0.02 | 8.58 | 0.00 | — | 0.09 | 0.83 |
| 0.25 | 0.50 | 0.19 | 0.79 | 0.13 | 1.19 | 0.22 | 0.76 |
| 0.50 | 1.00 | 0.49 | 0.50 | 0.35 | 0.66 | 0.56 | 0.49 |
| 1.00 | 2.00 | 0.63 | 0.73 | 0.56 | 0.56 | 0.76 | 0.59 |
| 2.00 | 4.00 | 0.71 | 1.21 | 0.66 | 0.73 | 0.79 | 1.08 |
| ratio 2:1 | | | | | | | |
| 0.20 | 0.10 | 0.00 | — | 0.00 | — | 0.07 | 0.78 |
| 0.50 | 0.25 | 0.30 | 0.66 | 0.17 | 0.97 | 0.48 | 0.41 |
| 1.00 | 0.50 | 0.60 | 0.71 | 0.50 | 0.43 | 0.69 | 0.56 |
| 0.00 | 1.00 | 0.70 | 1.71 | 0.66 | 0.46 | 0.80 | 0.90 |
| 4.00 | 2.00 | 0.79 | 1.91 | 0.75 | 0.61 | 0.82 | 1.72 |

TABLE 7
Average Fa for the products alone and standard deviation (SD)
calculated for all of the three experiments represented in Table 5.

| [NaBt] | [Met] | Average | |
|---|---|---|---|
| (mM) | (mM) | Fa | SD |
| 0.10 | — | 0.02 | 0.02 |
| 0.20 | — | 0.01 | 0.01 |
| 0.25 | — | 0.06 | 0.08 |
| 0.50 | — | 0.03 | 0.04 |
| 1.00 | — | 0.24 | 0.08 |
| 2.00 | — | 0.47 | 0.25 |
| 4.00 | — | 0.61 | 0.21 |
| — | 0.10 | 0.06 | 0.03 |
| — | 0.20 | 0.05 | 0.01 |
| — | 0.25 | 0.07 | 0.01 |
| — | 0.50 | 0.10 | 0.03 |
| — | 1.00 | 0.21 | 0.04 |
| — | 2.00 | 0.32 | 0.03 |
| | 4.00 | 0.41 | 0.05 |

TABLE 8
Average of Fa for the products in combination, standard deviation
(SD), and corresponding CI, calculated for all of the
three experiments represented in Table 6.

| | | | |
|---|---|---|---|
| [NaBt] | [Met] | Fa | CI |

| | | | | | |
|---|---|---|---|---|---|
| (mM) | (mM) | Average | SD | Average | SD |

ratio 1:1

| | | | | | |
|---|---|---|---|---|---|
| 0.10 | 0.10 | 0.02 | 0.01 | 2.82 | 1.50 |
| 0.25 | 0.25 | 0.10 | 0.05 | 1.30 | 0.53 |
| 0.50 | 0.50 | 0.37 | 0.08 | 0.57 | 0.07 |
| 1.00 | 1.00 | 0.62 | 0.11 | 0.59 | 0.11 |
| 2.00 | 2.00 | 0.70 | 0.08 | 0.95 | 0.34 |

ratio 1:2

| | | | | | |
|---|---|---|---|---|---|
| 0.10 | 0.20 | 0.06 | 0.05 | 4.70 | 5.48 |
| 0.25 | 0.50 | 0.18 | 0.05 | 0.92 | 0.24 |
| 0.50 | 1.00 | 0.47 | 0.11 | 0.55 | 0.09 |
| 1.00 | 2.00 | 0.65 | 0.10 | 0.62 | 0.09 |
| 2.00 | 4.00 | 0.72 | 0.07 | 1.01 | 0.25 |

ratio 2:1

| | | | | | |
|---|---|---|---|---|---|
| 0.20 | 0.10 | 0.04 | 0.05 | 0.78 | — |
| 0.50 | 0.25 | 0.32 | 0.16 | 0.68 | 0.28 |
| 1.00 | 0.50 | 0.60 | 0.10 | 0.57 | 0.14 |
| 0.00 | 1.00 | 0.72 | 0.07 | 0.84 | 0.36 |
| 4.00 | 2.00 | 0.79 | 0.04 | 1.41 | 0.70 |

The above results indicate that a synergy can be obtained on cell line Calu-6 when the products betaine citrate and metformin are combined at concentrations comprised between 0.5 and 1.5 mM of each of the products.

### Example 3

### Effect if Bromo-A lanine on Cancer Cell Lines Resistant to Taxol (U87-MG) in MTT Test

Cells of a line (U87-MG) intrinsically resistant to numerous drugs, in particular taxol, were incubated in the presence of a concentration of pure bromo-alanine of the order of 1 mM over a period of 72 hours.

These cultures resulted in a 40% inhibition of the growth of these cells with respect to the control comprising a standard culture medium.

### Example 4

### Effect of a Treatment Based on Chlorophosphoenol Pyruvate (Phosphoenol Pyruvate Carboxykinase Inhibitor) on the Mortality Caused by Cancer in Mice

Mice carrying a type L1210 intra-peritoneal lymphoma were treated for 7 days in parallel with the following preparations:
SYN857 (A): chlorophosphoenol pyruvate
SYN856 (B): chlorophosphoenol pyruvate and carnitine ester
BCNU: carmustine
Vehicle: control

The chlorophosphoenol pyruvate and the chlorophosphoenol pyruvate and carnitine ester were synthesized by Synthéval (Caen-France).

Carmustine is a drug commonly used in chemotherapy. It is used here as a positive control.

The weight of the animals for each group as well as the individual weight curves as a function of time were plotted and the mortality of the animals was monitored.

As can be noted from Table 9 hereafter:
the mice in the control group (vehicle) all died, with an average survival of 7.50±0.67 days (median 7 days);
4 mice out of 10 in the group treated with 136 mg/kg of chlorophosphoenol pyruvate (SYN857) had died at 9 days (T/C%>128%);
9 mice out of 10 in the group treated with 70 mg/kg of chlorophosphoenol pyruvate (SYN857) had died at 9 days (T/C%>128%);
6 mice out of 10 in the group treated with 60 mg/kg of chlorophosphoenol pyruvate and carnitine ester (SYN856) had died at 9 days (T/C%>128%),
7 mice out of 10 of the group treated with 30 mg/kg of SYN856 had died at 9 days, (T/C%>128%),
all the mice in the control group treated with 15 mg/kg of carmustine (BCNU) were still alive, which is normal.

The mice died as a result of the development of haemorrhagic ascites.

TABLE 9
Mortality observed in the different groups of mice.

| | | | | Average | | |
|---|---|---|---|---|---|---|
| | | | Number | number | Median | |
| | | Dose | of dead | of days of | value in | Ratio % |
| Groups | Treatment | (mg/kg/inj.) | mice | survival ± SD | days | Treated/controls |
| 1 | Vehicle | — | 10 | 7.50 ± 0.67 | 7 | |
| 2 | (SYN857) | 136 | 4 | 9.00 ± 00.0 | 9 | >128.5% |
| 3 | | 70 | 9 | 8.78 ± 0.42 | 9 | >128.5% |
| 4 | (SYN856) | 60 | 6 | 9.00 ± 0.00 | 9 | >128.5% |
| 5 | | 30 | 3 | 9.00 ± 0.00 | 9 | >128.5% |
| 6 | BCNU ® | 15 | 0 | NA | — | >128.5% |

The parameter T/C% is calculated with the ratio of the median survival time of the animals in the group considered to the median survival time of the animals in the control group, all multiplied by 100. In this case, it is significant as it is greater than 125% (>128% since not all the mice have yet died).

This attests to the anti-tumour effectiveness of the two preparations SYN857 (A) and SYN856 (B).

It is notable that the preparation SYN856 (B), which comprises the chlorophosphoenol pyruvate and carnitine ester in the form of a single molecule is effective at a lower concentration than chlorophosphoenol pyruvate alone.

1. Pharmaceutical composition intended for the treatment of cancer, comprising a combination of:
at least one PP2A methylating agent, and
at least one active ingredient including a PP1 phosphatase inhibitor, a histone deacetylase inhibitor, a direct or indirect pyruvate kinase activator, a PTEN agonist, a tyrosine kinase inhibitor, a PI3 kinase inhibitor, a glucose competitor, a phosphoenol pyruvate carboxykinase inhibitor, a citrate synthase inhibitor or a lactate dehydrogenase inhibitor.

2. Pharmaceutical composition according to claim 1, wherein the PP2A methylating agent is serine, folate, methionine, S-adenosyl methionine, vitamin B12, choline, acetylcholine manganochloride, betaine, or pharmaceutically acceptable salts thereof.

3. Pharmaceutical composition according to claims 1 wherein the PP1 phosphatase inhibitor is cantharidin, tautomycin, rapamycin or DARP 32.

4. Pharmaceutical composition according to claims 1, wherein the histone deacetylase inhibitor is butyrate, phenylbutyrate, trichostatin, or valproate.

5. Pharmaceutical composition according to claims 1, wherein the direct or indirect pyruvate kinase activator is xylulose 5-P, a ceramide, N-6-cyclopentyladenosine, or acetylcholine manganochioride.

6. Pharmaceutical composition according to claim 1, wherein the PTEN agonist is rosiglitazone.

7. Pharmaceutical composition according to claim 1, wherein the tyrosine kinase inhibitor is PD 9805 G or adenosine.

8. Pharmaceutical composition according to claims 1, wherein the glucose competitor is 5-mannhoseheptulose or 2-deoxy-glucose.

9. Pharmaceutical composition according to claim 1, wherein the PI3 kinase inhibitor is wortmannin, LY294002, quercetin, myricetin, staurosporine.

10. Pharmaceutical composition according to claim 1 wherein the phosphoenol pyruvate carboxykinase inhibitor is chlorophosphoenol pyruvate, aspartate, metformin or tryptophan derivatives.

11. Pharmaceutical composition according to claim 1, wherein the citrate synthase inhibitor is fluoro acetyl co-A.

12. Pharmaceutical composition according to claim 1, wherein the lactate dehydrogenase inhibitor is an alanine derivative.

13. Pharmaceutical composition according to claim 1, comprising the combination of a PP2A methylating agent and a PTEN agonist.

14. Pharmaceutical composition according to claim 13, comprising the combination of betaine citrate and rosiglitazone.

15. Pharmaceutical composition according to claim 1, comprising the combination of a PP2A methylating agent and a phosphoenol pyruvate carboxykinase inhibitor.

16. Pharmaceutical composition according to claim 15, comprising the combination of betaine citrate and metformin.

17. A method for treating cancer comprising administering the composition of claim 1 to a patient.

18. Product comprising at least one PP2A methylating agent and at least one active ingredient including a phosphatase inhibitor, a histone deacetylase inhibitor, a direct or indirect pyruvate kinase activator, a PTEN agonist, a tyrosine kinase inhibitor, a PTEN agonist, a tyrosine kinase inhibitor, a PI3 kinase inhibitor, a glucose competitor, a phosphoenol pyruvate carboxykinase inhibitor, a citrate synthase inhibitor or a lactate dehydrogenase inhibitor, as combination products for simultaneous or separate use or use spread over time in cytostatic or anti-cancer therapy.

19. Pharmaceutical composition according to claim 2, wherein the PP2A methylating agent is betaine citrate.

20. Pharmaceutical composition according to claim 12, wherein the lactate dehydrogenase inhibitor is bromo-alanine.
